(19) United States
(12) Patent Application Publication
(10) Pub. No.: US 2009/0246301 A1
(43) Pub. Date: Oct. 1, 2009

(54) METHOD FOR TREATING IRRITABLE BOWEL SYNDROME AND OTHER FUNCTIONAL GASTROINTESTINAL DISORDERS

(76) Inventors: Ben Z. Ehrenpreis, Skokie, IL (US); Eli D. Ehrenpreis, Skokie, IL (US)

Correspondence Address: JONATHAN D. FEUCHTWANG, 2912 BRITTAN AVE, SAN CARLOS, CA, 94070, US

(21) Appl. No.: 12/399,778

(22) Filed: Mar. 6, 2009

Related U.S. Application Data
(60) Provisional application No. 61/041,233, filed on Mar. 31, 2008.

(51) Int. Cl. A61K 36/534; A61P 1/00

(52) U.S. Cl. 424/747

(57) ABSTRACT

The invention relates to methods and compositions for the treatment of irritable bowel syndrome (IBS) and other functional gastrointestinal disorders. The methods of the invention involve the administration, in a delayed-release capsule or tablet containing a mixture of peppermint oil and chlorophyll. Other ingredients that may also be effective treatments may be included. This method may be useful as a new and safer treatment for IBS and other functional gastrointestinal disorders. The action of the peppermint oil is effective as a modulator of gastrointestinal motility and sensation. Chlorophyll may improve bowel activity by stimulation of secretion and motility. This is the first description of this unique mixture of these natural products for the treatment of gastrointestinal conditions.

### CLAIM OF PRIORITY

This Application claims priority from U.S. Provisional Patent Application Ser. No. 61/041,233 filed Mar. 31, 2008 entitled “METHOD FOR TREATING IRRITABLE BOWEL SYNDROME AND OTHER FUNCTIONAL GASTROINTESTINAL DISORDERS”

### FIELD OF THE INVENTION

Disclosed is a method to alleviate the major symptoms of a number of functional gastrointestinal disorders, particularly Irritable Bowel Syndrome (IBS).

### BACKGROUND OF THE INVENTION

Functional gastrointestinal disorders of the lower intestines are manifested by a variety of symptoms, including abdominal pain and cramping, constipation, diarrhea, abdominal bloating, gas production, early satiety and food intolerances. The most common functional gastrointestinal disorder is Irritable Bowel Syndrome (IBS), which has been found to affect up to 20% of the population of the United States. Functional gastrointestinal disorders can occur in childhood. Additionally, certain animals, such as cats, dogs, and horses may be affected.

Functional gastrointestinal disorders result in a large economic burden in the United States and other industrialized countries. For example, IBS patients have more than ten times as many overall physician visits for gastrointestinal complaints compared to the general population and also have more than twice the overall number of visits to physicians for non-gastrointestinal complaints. It has been estimated that the annual medical costs (including both direct and indirect costs) attributed to IBS in the USA are more than $20 billion per year.

Current basic and clinical research studies suggest that functional gastrointestinal disorders are associated with physiologic alterations of several organ systems: the gut, the spinal cord, and the brain. Altered signaling between these systems appears to be a major factor in the enhancement and perpetuation of these conditions. Just as the gastrointestinal tract sends signals to the brain in response to a variety of local stimuli, sensory input to the brain will affect the function of the gut. Integrated signaling occurs between the gut and the brain; these connected activities are influenced by a variety of factors. Increased intestinal motility and mucosal hyperemia induced by stressful or painful experiences are examples of alteration of gut function induced by the brain and spinal chord. Local gastrointestinal neurotransmitters, stored in the myenteric plexus, appear to play the most important role in these diseases, via regulation of normal and abnormal gastrointestinal function.

The most consistently demonstrated abnormality in patients with IBS is the presence of visceral hyperalgesia. For example, studies in patients with IBS show that these patients exhibit a markedly decreased threshold for the sensation of painful rectal stimuli, compared to normal controls. This phenomenon appears, in part, to be due to an abnormal function of local gastrointestinal serotonin (5HT-3) receptors, but may be influenced by altered gastrointestinal adrenergic, opioid, or neurokinin receptors, to name a few.

Many patients with IBS have abdominal discomfort, bloating and constipation. Patients with IBS and constipation are termed Constipation-Predominant IBS patients.

Limited treatment options are available for patients with IBS. Such patients and those with other functional gastrointestinal disorders, often have multiple symptoms occurring simultaneously, such as nausea, abdominal pain, and diarrhea. Because of this, individual patients with IBS are treated with multiple symptom-directed medications. Prescription medications for IBS include antispasmodic agents, anti-diarrheals, and sedatives (e.g. benzodiazepines). All of these have systemic side effects that limit their usefulness. Additionally, each one is primarily directed at single symptoms of these disorders.

The 5-HT4 receptor antagonist, Tegaserod (Zelnorm), was recently approved by the FDA for treatment of constipation-predominant IBS. It was subsequently removed from the market due to a higher than expected rate of cardiac events noted with patients taking this medication compared to placebo in post marketing studies. This drug, which directly binds the 5-HT4 receptors in the gastrointestinal tract with high-affinity, has been shown in laboratory studies to stimulate intestinal peristalsis and secretion and to reduce visceral sensitivity. Tegaserod and alosetron (Lotronex), a 5-HT3 receptor antagonist, work directly at the level of a local gastrointestinal neurotransmitter. Lotronex was removed from the general market due to the development of severe constipation and ischemic colitis. It is currently available on a limited basis only. At present, topical gastrointestinal therapy using delayed-release capsules has primarily been applied for the treatment of Inflammatory Bowel Disease, (ulcerative colitis and Crohn's Disease). Drugs in delayed-release forms, specifically designed to have a topical effect that are utilized for these conditions include mesalamine and budesonide.

Mesalamine (or 5-ASA) is an anti-inflammatory agent specifically administered as a topical drug, to reduce mucosal inflammation in these diseases. Several forms of mesalamine in delayed-release form are available. 5-ASA is administered in a delayed-release form for two purposes: 1) to ensure that reaches the affected area of the bowel, 2) to produce a topical effect of the drug at the site of release. One form of the drug, Asacol, consists of 5-ASA in a pH-dependent capsule (Eudragit-S), that releases the drug at a pH of 7, i.e., that of the distal ileum and colon. European forms of mesalamine (Claversal, Mesasal, and Salofalk) contain 5-ASA in a capsule that releases the active drug at a pH of 5 to 6.

Budensonide is a potent corticosteroid agent that exhibits both powerful anti-inflammatory effects and rapid inactivation by the liver (termed first-pass metabolism). These properties make budesonide an attractive agent for the treatment of IBD. Entocort is a new form of budesonide in a delayed-release capsule that dissolves at a pH of 5.5 or greater. This promotes the topical effect of the drug in the distal small intestine; the drug is then rapidly destroyed by the liver after absorption, thus preventing many of the side effects that occur with systemic administration of other steroids.

The gastrointestinal effects of peppermint oil have been studied in animal and humans. Peppermint oil contains menthol, a smooth muscle relaxant. It also contains menthone, cineol, and several other volatile oils. Animal studies demonstrate that peppermint oil relaxes intestinal smooth muscle. This effect may occur from antagonistic effect of peppermint oil on gastrointestinal calcium channels. The smooth muscle relaxation that is produced by menthol occurs as a direct topical effect. Due to these properties of menthol, peppermint oil administration results in relaxation of the lower esophageal sphincter, and ingestion of peppermint oil in a liquid form produces symptoms of gastroesophageal reflux. To avoid the development of gastroesophageal reflux, peppermint oil may be administered in a delayed-release capsule. This form of administration of peppermint oil has been studied for the treatment of irritable bowel syndrome (IBS). A meta-analysis involving 175 patients from five clinical trials showed a statistically significant relief of symptoms of IBS in patients treated with peppermint oil compared with those treated with placebo. Two additional more recent studies confirmed the efficacy of peppermint oil for the treatment of IBS. A single study also showed that a combination of peppermint oil and caraway oil was effective for treatment of non-ulcer dyspepsia. Chlorophyll, the pigment utilized by plants to facilitate the conversion of carbon dioxide (CO2) to oxygen (O2) and create energy, has been used as a remedy for a variety of conditions. It has been used as a folk remedy for halitosis, pancreatitis, reduction of colostomy odor and constipation. These effects have not been proven through scientific studies. No studies of the mechanisms of these effects have been performed. Chlorophyllin, the absorbable portion of chlorophyll has been used to reduce body odors. Only limited studies have been performed on chlorophyllin. This combination alone or together with other natural ingredients has not been studied and is not available in the US market. Liquid chlorophyll is available that is flavored with nonpharmacologic amounts of peppermint oil to make it palatable for ingestion.

### SUMMARY OF THE INVENTION

Disclosed is a method of treating functional gastrointestinal disorders, including the steps of providing a delayed-release capsule containing a pharmacologic dose of peppermint oil combined with at least one of chlorophyll and chlorophylline; and orally administering the delayed-release capsule, wherein the delayed-release capsule delivers the peppermint oil and chlorophyll to the small intestine and/or colon.

According to one embodiment, the functional gastrointestinal disorder is irritable bowel syndrome.

According to another embodiment, the functional gastrointestinal disorder is chronic functional constipation.

According to another embodiment, the functional gastrointestinal disorder is functional dyspepsia.

According to another embodiment, the disorder is functional abdominal pain.

According to another embodiment, the disorder is functional abdominal bloating.

According to another embodiment, the functional gastrointestinal disorder is pelvic outlet disorder.

According to another embodiment, the functional gastrointestinal disorder is intestinal gas.

In each of the aforementioned embodiments, the delayed-release capsule may be a pH-sensitive capsule.

In each of the aforementioned embodiments, the delayed-release capsule may be an enteric coated capsule.

In each of the aforementioned embodiments, the delayed-release capsule may be EUDRAGIT L.

In each of the aforementioned embodiments, the delayed-release capsule may be EUDRAGIT R.

In each of the aforementioned embodiments, the delayed-release capsule may be EUDRAGIT S.

In each of the aforementioned embodiments, the delayed-release capsule may a delayed release tablet.

In each of the aforementioned embodiments, the dose of peppermint oil may be between 0.05 to 4 mL per capsule.

In each of the aforementioned embodiments, the dose of chlorophyll or chlorophylline may be between 5 and 200 mg.

In each of the aforementioned embodiments, the total dose of contained ingredients may be between 1 mg to 1000 mg.

### DETAILED DESCRIPTION OF THE INVENTION

The present embodiment presents methods for delivering peppermint oil and chlorophyll or chlorophyllin to the small and/or large intestine via a delayed release system or a slow release tablet. In so doing, concentrations of these ingredients will be sufficiently high to effect the desired therapeutic actions, while limiting gastroesophageal reflux developing from peppermint oil exposure in the stomach and esophagus. Additional ingredients such as caraway oil may be added to improve antispasmotic effects of the product for treating IBS and other gastrointestinal disorders.

Several methods are used for this purpose. First, the present invention uses peppermint oil, a natural treatment shown to be effective for IBS and other gastrointestinal disorders. Second, the present invention combines peppermint oil with chlorophyll and or chlorophyllin, natural substances that may improve other gastrointestinal symptoms including constipation. The method also allows for the incorporation of additional substances, such as caraway oil that may effective for the treatment of IBS and other gastrointestinal symptoms. Finally, the invention utilizes a system to deliver these ingredients directly to the sites of action in the small and large intestines, thereby avoiding release of theses ingredients in the stomach. This enables the contents to reach their target at high concentration and to avoid local effects of the peppermint oil in the stomach.

Both peppermint oil and chlorophyll have potential for the treatment of intestinal gas. Peppermint oil may reduce bloating sensations associated with gas buildup via relaxation of the intestinal smooth muscle. Peppermint oil holds potential as a fecal deodorant. Chlorophyll and chlorophyllin may function as fecal deodorants.

Theoretically, other drugs including laxatives and modulators of gastrointestinal function could be used for the intended purposes in place of these ingredients. However, use of a combination of peppermint oil, chlorophyll and/or chlorophyllin, with the possible addition of other natural ingredients has advantage over other drugs for IBS and other gastrointestinal conditions, namely, greatly reduced or even absence of significant systemic adverse reactions. Chlorophyll has little systemic absorption. Toxicity of chlorophyll and chlorophyllin appears to be limited to photosensitivity. Peppermint oil at the doses employed has been widely used and recognized as safe.

Peppermint oil, chrolophyll, chlorophyllin and caraway oil are all available individually as over-the-counter dietary supplements. A combination of peppermint and caraway oils has been used for the treatment of non-ulcer dyspepsia. This combination is not available in the US market. To date, no combination of peppermint oil and chlorophyll and/or chlorophyllin have been developed for the treatment of gastrointestinal diseases. This combination alone or together with other natural ingredients has not been studied. Small, non-pharmacologic amounts of peppermint oil has been combined with liquid chlorophyll for the purpose of flavoring the chlorophyll to make it more palatable. Therefore some liquid chlorophyll preparations are sold that are peppermint flavored due to adding small quantities of peppermint oil.

The inventors have been able to show that peppermint oil may be mixed without the requirement of heat with chlorophyll copper complex. This forms a uniform substance with an oily consistency with complete melding of the two individual ingredients. To date, this combined substance has not been previously described in a capsule, delayed release capsule or tablet form. To date, this combined substance at concentrations of both ingredients at pharmacologic doses has not been previously described.

### Clinical Experience:

The authors have administered the combination of peppermint oil in a delayed released capsule form and chlorophyll copper complex as combination therapy in four patients with IBS, bloating, constipation and gas. All developed improvement in their condition from the combination treatment.

The basic premise of the present invention is to deliver, in a controlled manner, a combination of peppermint oil and chlorophyll. The combination of these natural substances hold promise for the treatment of IBS and other gastrointestinal disorders via their action on gastrointestinal smooth muscle and laxative effects. Other natural substances may be added to enhance the effectiveness of the combination, including but not limited to caraway oil, lemon balm, 1-tryptophan, hops oil, d,l phenylalanine, SAMe, valerian root, bismuth, etc. It is assumed that the essential oils or liquid forms of these will be used for the product. By means of a pH-dependent delivery system, delayed release capsule that resists breakdown in the stomach or delayed release tablet, orally-administered product can be delivered to sites in the gastrointestinal tract from which many of these disorders originate; the small and large intestines. Once they arrive at these sites, the ingredients will have a topical effect and in this way, alleviate many of the symptoms of the diseases, namely, constipation, dyspepsia, abdominal pain, bloating, cramping, etc.

The individual ingredients of the product have been available for some time, as food supplements and natural remedies. They have a long and established safety record. However, the combination of these ingredients has not been disclosed or suggested by literature. According to a first embodiment, peppermint oil and chlorophyll and/or chlorophylline are provided in a time-delayed capsule. The content of peppermint oil in each capsule is between 0.05 and 0.4 mL. The amount of chlorophyll and chlorophylline in each capsule is between 5 and 200 mg. Optionally, the time-delayed capsule may further contain between 1 to 200 mg of caraway oil. The time-delayed capsule may be an acid-resistant capsule or tablet, e.g., enteric coated capsules or tablets, EUDRAGIT-L, EUDRAGIT R; the total amount of active ingredients will range from 6 milligrams to 2 grams/capsule. Doses administered to patients with IBS or other disorders vary from 5 milligrams to 20 grams per day. The capsules or tablets are swallowed with water or other liquid vehicle, e.g., juice, or milk. These doses will be taken up to four times daily.

The optimal capsule and tablet sizes and dosages are determined by the results of preliminary studies and can vary with the age, size, and weight of the subject (patient).

The invention also contemplates embedding the active ingredient in a delayed release capsule such as EUDRAGIT L or EUDRAGIT R, which is resistant to acid pH also, but releases the agent in a neutral or alkaline pH. Alternatively, the active ingredient is presented in a particulate form in which the particles are covered by a coating that can only be removed or dissolved in non-acidic conditions. In some of the embodiments of the invention, the marker agent may be covered by a polymeric agent that is resistant to acidic pH.

Additionally, the invention encompasses the placement of peppermint oil combined with chlorophyll and/or chlorophyllin possibly with additional ingredients as described in other delayed-release forms of capsules, including azopolymers that resist breakdown, until exposure to colonic bacteria. The invention also includes the use of these ingredients in enteric-coated tablets that resist gastric degradation. Active product release from enteric-coated tablets is delayed until the tablets are emptied from the stomach. The invention includes tablet coatings, such as cellulose acetate phthalate, copolymer poly and other polymers utilized for this purpose.

The pores formed by the polymeric components of such microspheres shrink in the stomach (i.e., at low pH), thereby preventing the release of the encapsulated active agent. Once the microspheres pass into the small intestine, where the pH tends toward neutral (i.e., about pH 6.0 and higher), the pores of the microspheres swell, thereby releasing the entrapped marker agent. The swelling/shrinking phenomenon is referred to as complexation (Lowman and Peppas, Macromolecules, 30 (1997) pp. 4959-4965). Once the peppermint oil and chlorophyll (as well as other potential ingredients) are released, they remain essentially confined to the gastrointestinal tract.

The preparation of pH-sensitive microparticles is known to those of skill in the art (Lowman et al, in: Tailored Polymeric Materials for Controlled Delivery Systems. I. McCullough and S. Shalaby (Eds.), ACS, Washington, DC, ACS Symposium Series, 709, 1998, pp. 156-164). In exemplary embodiments, the active ingredient is dissolved in an appropriate solvent, e.g., ethanol or ethanol solutions of various concentration in which the pH of the solution is alkaline. Dry copolymer microparticles are dispersed in a solution of the desired marker agent and stirred at a constant rate for one day. The alkaline solution causes the microparticles to swell and the marker agent is taken up into the pores of the microparticles. The weight ratio of the marker agent to polymer in the initial solution may be varied, e.g., from 1:1 to 1:6. Following marker loading, the solutions are filtered using 1 mm filter paper and an equal volume of an acid fluid is added to de-swell the marker-loaded particles. These hydrogels containing the marker of interest are then dried in vacuo for three days and stored at 4° C. prior to use.

In alternative embodiments, the ingredients may be formulated such that it is coated with an enteric coating which is resistant to dissolution in acidic conditions. Preferably, the coating is such that the enteric coating is predisposed to dissolution in the middle and distal portions of the small intestine (see U.S. Pat. No. 5,795,882, incorporated herein by reference). One widely used enteric film coating system is Eudragit. Processing of EUDRAGIT may also involve additional excipients such as plasticizers that decrease the minimum film forming temperatures and the glass transition temperatures. Adding a plasticizer such as triethyl citrate improves the flexibility of the film coatings. Glidants like glycerol monostearate or talc may be added to prevent the film coatings from becoming sticky. Pigments may also be incorporated or bound into the film coating. A variety of Eudragit enteric coating products may be used including EUDRAGIT L 30 D-55, EUDRAGIT L 100-55, EUDRAGIT L 100, EUDRAGIT L S 10o, EUDRAGIT R and EUDRAGIT S.

As used herein, “pharmaceutically acceptable carrier” includes any and all solvents, dispersion media, coatings, antibacterial and antifungal agents, isotonic, and absorption-delaying agents and the like. The use of such media and agents for pharmaceutically-active substances is well known in the art. Except insofar as any conventional media or agent is compatible with the active ingredient, its use in the therapeutic compositions is contemplated. Supplementary active ingredients also can be incorporated into the compositions.

The compositions and/or methods disclosed and claimed herein can be made and executed without undue experimentation, in light of the present disclosure.

While the compositions and methods of this invention have been described in terms of preferred embodiments, it will be apparent to those of skill in the art that variations may be applied to the compositions and/or methods and in the steps or in the sequence of the steps of the method described herein, without departing from the concept, spirit, and scope of the invention. More specifically, it will be apparent that certain agents which are both chemically and physiologically related may be substituted for the agents described herein, while the same or similar results would be achieved. All such similar substitutes and modifications apparent to those skilled in the art are deemed to be within the spirit, scope, and concept of the invention, as defined by the appended claims.

The references cited herein throughout to the extent that they provide exemplary procedural or other details supplementary to those set forth herein, are all incorporated herein by reference.

What is claimed is:

1. A method of treating functional gastrointestinal disorders, comprising:
a. Providing a delayed-release capsule containing a pharmacologic dose of peppermint oil combined with at least one of chlorophyll and chlorophylline; and
b. Orally administering the delayed-release capsule, wherein the delayed-release capsule delivers the peppermint oil and chlorophyll to the small intestine and/or colon.

2. The method of claim 1, wherein the functional gastrointestinal disorder is irritable bowel syndrome.

3. The method of claim 1, where the functional gastrointestinal disorder is chronic functional constipation.

4. The method of claim 1, where the functional gastrointestinal disorder is functional dyspepsia.

5. The method of claim 1, where the disorder is functional abdominal pain.

6. The method of claim 1, where the disorder is functional abdominal bloating.

7. The method of claim 1, where the functional gastrointestinal disorder is pelvic outlet disorder.

8. The method of claim 1, where the functional gastrointestinal disorder is intestinal gas.

9. The method of claim 1 in which the delayed-release capsule is a pH-sensitive capsule.

10. The method of claim 6 in which the delayed-release capsule is an enteric coated capsule.

11. The method of claim 6 in which the delayed-release capsule is EUDRAGIT L.

12. The method of claim 6 in which the delayed-release capsule is EUDRAGIT R.

13. The method of claim 6 in which the delayed-release capsule is EUDRAGIT S.

14. The method of claim 6 in which the delayed-release capsule is a delayed release tablet.

15. The method of claim 1 in which the dose of peppermint oil is between 0.05 to 4 mL per capsule.

16. The method of claim 1 in which the dose of chlorophyll or chlorophylline is between 5 and 200 mg.

17. The method of claim 1 in which the total dose of contained ingredients is 1 mg to 1000 mg.
